(19) United States
(12) Patent Application Publication
(10) Pub. No.: US 2011/0029047 A1
(43) Pub. Date: Feb. 3, 2011

(54) HEATING ELEMENT

(75) Inventors: Tetsuya Maruyama, Ibaraki-shi (JP); Atsushi Matsuo, Osaka-shi (JP)

Correspondence Address: DAY PITNEY LLP, 7 TIMES SQUARE, NEW YORK, NY, 10036-7311, US

(73) Assignee: KOBAYASHI PHARMACEUTICAL CO., LTD., Osaka-shi, Osaka (JP)

(21) Appl. No.: 12/934,890

(22) PCT Filed: Feb. 19, 2009
(86) PCT No.: PCT/JP2009/052865

§ 371 Date: Sep. 27, 2010

(30) Foreign Application Priority Data
Mar. 31, 2008 (JP) 2008-92911

(51) Int. Cl. A61F 7/00

(52) U.S. Cl. 607/96

(57) ABSTRACT

A heating element has an exothermic composition that generates heat by contact with air. The exothermic composition is sealed in an air-permeable bag that has an outer face made of a fibrous sheet. The air-permeable bag is formed of a laminate obtained by layering a resin film on the fibrous sheet. The laminate is formed by discontinuously bonding the fibrous sheet and the resin film. The filing rate of the exothermic composition ranges from 4 to 95%.

### TECHNICAL FIELD

The present invention relates to a heating element, particularly to a heating element used as an instrument for treatment or rehabilitation.

### BACKGROUND ART

Various instruments for the training or rehabilitation of fingers, hands etc. have been suggested. For example, Patent Documents 1 and 2 disclose a finger-training instrument having a recessed finger pull.

However, the instruments disclosed in Patent Documents 1 and 2 apply an excessive load to the hands and fingers during training. Therefore, the instruments allow the user to perform training only for a limited time.

For the treatment or rehabilitation of arthralgia in hands, fingers, arms etc., a method of heating the affected part with a heating element has been adopted. In particular, in the treatment or rehabilitation of arthralgia in fingers, an exercise of grasping and releasing a heating element is performed as the fingers are heated with the heating element. As an example of such a heating element, Patent Document 3 discloses a heating element having a substantially round shape, containing an exothermic composition that generates heat by contact with air.

However, since the heating element of Patent Document 3 is fabricated by sealing an exothermic composition in a bag that is used for a general heating element, the heating element does not completely fit into the hand; therefore, this heating element is not adequate for the exercise of grasping and releasing the heating element. Accordingly, the heating element of Patent Document 3 allows the user to perform the exercise only for a limited time, decreasing the efficiency of the treatment or rehabilitation.

Patent Document 4 discloses another example of an instrument for the above exercise. This instrument, for use in the exercise of hands and fingers, is fabricated by covering the surface of a gel material with a rubbery film.

However, the exercise instrument of Patent Document 4 is too hard to fit into a hand. Therefore, when a user performs an exercise with the instrument of Patent Document 4, an excessive load is applied to the hand, thereby only allowing the user to perform the exercise for a limited time. Moreover, since the instrument of Patent Document 4 is not capable of giving a thermal effect, it is not suitable for the treatment or rehabilitation of arthralgia in hands etc.

Patent Document 5 discloses a heat cell serving as an instrument for treating temporary or chronic pains. The heat cell contains an exothermic composition that generates heat by contact with air.

However, the heat cell of Patent Document 5 is adapted to be adhered to a knee, the head, the back or the like, and cannot serve as a treatment or rehabilitation instrument for arthralgia in hands or the like.

Therefore, there has been demand for the development of a heating element that allows the user to appropriately perform an exercise of grasping and releasing the heating element with a hand for a long period of time, while heating the affected part.

### [Citation List]

### [Patent Document 1] Japanese Unexamined Patent Publication No. 1996-336616

### [Patent Document 2] Japanese Unexamined Patent Publication No.

### [Patent Document 3] Japanese Unexamined Patent Publication No. 1995-63493

### [Patent Document 4] Japanese Utility Model Publication No. 1994-75528

### [Patent Document 5] Japanese Unexamined Patent Publication No. 1999-508786

### SUMMARY OF INVENTION

### Technical Problem

An object of the present invention is to provide a heating element allowing the user to appropriately perform an exercise of grasping and releasing the heating element with a hand for a long period of time, while heating the affected part.

### Solution to Problem

The inventors of the present invention conducted extensive research, and found that the above object can be achieved using a heating element fabricated by filling a bag having a specific structure with a predetermined amount of an exothermic composition that generates heat by contact with air. With this finding, the inventors completed the present invention.

Specifically, the present invention relates to the following heating elements.

Item 1. A heating element comprising an exothermic composition that generates heat by contact with air, the exothermic composition being sealed in an air-permeable bag that has an outer face made of a fibrous sheet,

wherein:
(1) the air-permeable bag is formed of a laminate obtained by layering a resin film on the fibrous sheet,
(2) the laminate is formed by discontinuously bonding the fibrous sheet and the resin film, and
(3) the filing rate of the exothermic composition ranges from 4 to 95%.
   Item 2. The heating element according to Item 1, wherein the fibrous sheet and the resin film in the laminate are bonded at an adhesion strength of from 0.2 to 6 N.
   Item 3. The heating element according to Item 1 or 2, wherein an adhesive layer is discontinuously formed between the fibrous sheet and the resin film.
   Item 4. The heating element according to any one of Items 1 to 3, wherein the air-permeable bag has a spherical or cylindrical shape.
   Item 5. The heating element according to any one of Items 1 to 4, wherein the fibrous sheet is a nonwoven or woven cloth of natural or artificial fiber.
   Item 6. The heating element according to any one of Items 1 to 5, wherein the exothermic composition is a composition containing iron powder, activated carbon, metal salt and water, and wherein the contents of the iron powder, the activated carbon, the metal salt and the water are 30 to 80 mass %, 3 to 25 mass %, 0.5 to 10 mass % and 1 to 40 mass %, respectively.
   Item 7. The heating element according to any one of Items 1 to 6, wherein the resin film contains, as a resin component, at least one synthetic resin selected from the group consisting of polyethylene, polypropylene, nylon polyester and polyvinyl chloride.
   Item 8. The heating element according to any one of Items 1 to 7, wherein the heating element is an instrument for treatment or rehabilitation.

The heating element of the present invention comprises an exothermic composition that generates heat by contact with air, the exothermic composition being sealed in an air-permeable bag that has an outer face made of a fibrous sheet,

wherein:
(1) the air-permeable bag is formed of a laminate obtained by layering a resin film on the fibrous sheet,
(2) the laminate is formed by discontinuously bonding the fibrous sheet and the resin film, and
(3) the filing rate of the exothermic composition ranges from 4 to 95%.

In the heating element of the present invention, the exothermic composition is contained in a bag made of a laminate of a fibrous sheet and a resin film that are discontinuously bonded. The discontinuous bond of the fibrous sheet and the resin film allows the heating element to fit into the hand when the user grasps the heating element.

In the present specification, the phrase “fit into a hand” describes a state where the fingers of the hand grasping the heating element with an appropriate amount of strength can be pressed into the heating element, and the hand can sense a moderate repulsive force when the fingers pressed into the heating element are relaxed.

The heating element of the present invention fits into the hand grasping the heating element, thereby allowing the user to continuously perform an exercise of grasping and releasing the element with a hand for a long period of time. Moreover, the heating element of the present invention appropriately exhibits a thermal effect.

In the present specification, the phrase “thermal effect” refers to an effect of warming the affected part by generating heat. The thermal effect facilitates, for example, an increase in blood circulation in the affected part, removal of metabolic decomposition products from the tissue in the affected part, and recovery of the affected part.

The heating element of the present invention allows the user to continuously perform an exercise of grasping and releasing the element with a hand for a long period of time, and also appropriately exhibits a thermal effect during the exercise. Therefore, the heating element of the present invention is most appropriate for instruments for treatment or rehabilitation of arthralgia in fingers etc.

### Bag

The bag as a component of the heating element of the present invention is air-permeable. The heating element of the present invention generates heat such that outside air is introduced into the bag so as to contact the exothermic composition with air, causing the exothermic composition to generate heat.

The bag is constituted of a laminate having an outer face made of a fibrous sheet. A resin film is layered on the fibrous sheet. More specifically, the bag has a multilayer structure in which the outer face, which comes in contact with a hand, is made of a fibrous sheet, and an inner face, which does not come in contact with a hand but rather with the exothermic composition, is made of a resin film.

Examples of the fibrous sheet include woven and nonwoven fabrics of natural fibers or synthetic fibers. Examples of natural fibers include cotton, linen, silk and wool. Examples of synthetic fibers include nylon, polyethylene, polypropylene, polyester, polyvinyl chloride, vinylon, rayon, and acryl. Particularly preferable fibrous sheets among them are woven or nonwoven nylon fabrics that can improve the fitting of the heating element into a hand.

The air-permeability of the fibrous sheet is not limited insofar as a sufficient amount of air can be introduced into the bag.

The thickness of the fibrous sheet is not limited insofar as the heating element can sufficiently fit into a hand and the thermal effect can be appropriately exhibited. The thickness is generally in a range of from about 100 to 350 μm, preferably in a range of from about 200 to 300 μm.

The resin film preferably contains a synthetic resin such as polyethylene, polypropylene, nylon, polyester, polyvinyl chloride or the like, as a resin component. These synthetic resins may be used solely, or in a combination of two or more. It is particularly preferable to use a resin film containing polyethylene as a resin component. In addition to the resin component, the resin film may contain various additives such as antioxidants, UV absorbers, dyes, pigments, antistatic agents, nucleating agents etc., insofar as the effect of the present invention is not impaired.

The resin film is air-permeable. Examples of the resin films include a film obtained by adding an inorganic filler, such as calcium carbonate, and liquid paraffin to the resin component, and extending the resulting resin composition; an air-permeable resin film provided with air-permeable pores that were formed using a needle, laser, electron discharge method or the like; and a microporous film that originally has a large number of micropores as air-permeable pores.

The air-permeability of the resin film can be adjusted by appropriately setting the size, number etc. of the air-permeable pores of the resin film depending on the type, the amount, the temperature of the heat or the like of the exothermic composition. Since the fibrous sheet has sufficient air permeability, the air-permeability of the bag substantially depends on the pore diameter of the air-permeable pores of the resin film.

The air-permeability of the resin film can be confirmed by measuring the air-permeability rate. The water vapor transmission rate of the bag is usually about 100 to 1,000 g/m²·day, preferably about 200 to 600 g/m²·day, more preferably about 300 to 400 g/m²·day.

The water vapor transmission rate disclosed in the present specification was measured according to “Testing methods for determination of the water vapor transmission rate of moisture-proof packaging materials (dish method)” defined by JIS Z0208.

The thickness of the resin film is determined within a range that does not impair the effect of the present invention; specifically, the thickness is generally about 5 to 200 μm, preferably about 50 to 100 μm.

The laminate is obtained by discontinuously bonding the fibrous sheet and the resin film. This discontinuous bond of the fibrous sheet and the resin film improves the feel of use when the user grasps the heating element with a hand. More specifically, the discontinuous bond allows the heating element to sufficiently fit into the hand.

Here, “discontinuous bond” refers to a state where the fibrous sheet and the resin film are not continuously bonded, but are bonded via irregularly provided adhesive portions and non-adhesive portions. More specifically, the adhesive portion, through which the fibrous sheet is bonded with the resin film, is not provided over the entire area, but only on a part of the fibrous sheet.

The adhesion strength of the fibrous sheet and the resin film to form the laminate is preferably about 0.2 to 6 N, more preferably about 0.3 to 3 N. The present invention is not strictly limited to this range; however, it is assumed that an adhesion strength of about 0.2 to 6 N allows a part of the adhesion portion of the resin film to be easily removed from the fibrous sheet when the heating element of the present invention is used. This allows the heating element to appropriately fit into the hand.

The adhesion strength can be measured according to a 180° peeling resistance test defined by JIS Z0237.

Particularly, in the laminate, the area of the portion where the fibrous sheet and the resin film are adhered to each other is preferably about 30 to 90%, more preferably about 45 to 75% of the entire area of the fibrous sheet.

The fibrous sheet and the resin film may be bonded such that the fibrous sheet and the resin film are bonded via an adhesive layer, or are directly bonded without an adhesive layer. The former state using an adhesive layer is more preferable as the laminate.

The adhesive layer is formed, for example, by applying a resin component on the fibrous sheet. The resin component is preferably selected from various flexible resins. Examples of flexible resins include vinyl acetate, polyvinyl acetal, ethylene vinyl acetate, vinyl chloride, acryl, polyamide resins, cellulose resins, and α-olefin resins. These resins may be used solely, or in combination. Ethylene vinyl acetate resin is preferable.

The application of the resin component can be carried out using suitable known methods. A method using a spray gun is preferable. The application method using a spray gun allows for formation of a desirable discontinuous adhesive layer.

The application amount of the resin component is determined within a range that ensures desired formation of the discontinuous adhesive layer and target adhesion strength.

The thickness of the adhesive layer is not particularly limited insofar as the heating element can sufficiently fit into a hand. The thickness is generally in a range of from about 1 to 50 μm, preferably about 1 to 20 μm.

As an example of the method of directly bonding the fibrous sheet and the resin film without using an adhesive layer, a method of bonding the fibrous sheet and the resin film using thermocompression bonding can be adopted. Thermocompression bonding adheres the fibrous sheet and the resin film with each other by applying heat using a heating means, and also applying pressure as necessary. Examples of the heating means include heat sealers, heat rolls, hot air etc.

In the thermocompression bonding method, the fibrous sheet and the resin film are bonded under appropriate heating conditions, pressure conditions etc. For example, when the thermocompression bonding is performed using a heat sealer, the sealing temperature is preferably about 60 to 150° C. The sealing time is preferably about 0.5 to 10 seconds.

The bag is formed into a shape that allows the user to appropriately perform an exercise of grasping and releasing the heating element. Particularly, the bag preferably has a spherical or cylindrical shape. The “spherical shape” is not limited to a perfect sphere, but may be a substantially spherical shape such as an egg shape, conical shape etc. A drawstring bag shape is particularly preferable for the heating element of the present invention.

As shown in FIG. 1, a “drawstring bag” refers to a spherical container with an opening tied with a string.

The capacity of the bag is not particularly limited insofar as the bag can contain the exothermic composition (described later).

### Exothermic Composition

The exothermic composition to be sealed in the bag may be selected from known exothermic compositions that generate heat by contact with air. The present invention particularly favors a composition containing iron powder, activated carbon, metal salt and water.

The total amount of iron powder, activated carbon, metal salt and water in the exothermic composition is preferably about 80 to 100 mass %.

The following more specifically explains the above-described exothermic composition in reference to a typical exothermic composition that contains iron powder, activated carbon, metal salt and water.

### Iron Powder

The heating element of the present invention exhibits a thermal effect by the heat generated from the reaction of the iron powder with oxygen in the air.

Examples of iron powder include reduced iron and cast iron. They can be used solely, or in a combination of two or more.

The iron powder may be grains or fibers. It is possible to solely use either grains or fibers; otherwise, a mixture of grains and fibers may be used. Granular iron powder is particularly preferable because it allows the heating element to more appropriately fit into the hand.

The grain diameter of the iron powder is determined within a range that allows the heating element to fit into the hand, and that ensures an appropriate thermal effect. The grain diameter is generally about 10 to 300 μm, preferably about 10 to 100 μm.

The grain diameter specified in the present specification is calculated by screening 100 g of the target sample (iron powder etc.) using an electric vibrating screen that contains sieves of 700 μm, 650 μm, 500 μm, 400 μm, 300 μm, 250 μm, 100 μm, 50 μm, and 10 μm from the top to the bottom of the screen. The screening is performed by turning on the screen for 15 minutes, and measuring the amount of grains that pass through each sieve, and the amount of grains that remain on the sieve.

The content of the iron powder in the exothermic composition is preferably about 30 to 80 mass %, more preferably about 45 to 65 mass %.

### Activated Carbon

An activated carbon serves to facilitate air supply by capturing air in the micropores in its surface, and also maintain the thermal effect to keep the heat liberation temperature constant. The internal structure of the activated carbon is highly porous, and therefore has a remarkably excellent water retention property. Further, in addition to the excellent water absorption, activated carbon also absorbs vapor resulting from the heat generation from the exothermic composition, thereby suppressing discharge of vapor. Thus, an activated carbon also serves as a water retention substance. An activated carbon further serves to absorb odor caused by oxidation of iron powder.

Examples of suitable activated carbons include activated carbons prepared from coconut shell, wood, charcoal, mineral coal, bone coal etc.

The suitable form of the activated carbon is, for example, grains, or fibers. It is possible to solely use either grains or fibers; otherwise, a mixture of grains and fibers may be used. Granular activated carbon is particularly preferable in the present invention because it allows the heating element to more appropriately fit into the hand.

The grain diameter of the granular activated carbon is set in a range that allows the heating element to fit into the hand, and that ensures an appropriate thermal effect. The grain diameter is generally about 10 to 300 μm, preferably about 10 to 100 μm.

The content of the activated carbon in the exothermic composition is preferably about 3 to 25 mass %, more preferably about 10 to 20 mass %.

### Metal Salt

Metal salt serves to, for example, activate the surface of the iron powder to enable the iron powder to more easily undergo oxidative reaction with air, thereby facilitating the oxidative reaction of the iron.

The metal salt may be selected from various metal salts used for known exothermic compositions. Examples of metal salts include sulfates such as ferric sulfate, potassium sulfate, sodium sulfate, or manganese sulfate; and chlorides such as cupric chloride, potassium chloride, sodium chloride, calcium chloride, manganese chloride, magnesium chloride, or cuprous chloride. In addition, carbonates, acetates, nitrates and other salts can also be used. These mineral salts may be used solely, or in combination.

The particle diameter of the metal salt is set within a range that does not impair the effect of the present invention. The particle diameter is generally about 100 to 700 μm, preferably about 250 to 650 μm.

The content of the metal salt in the exothermic composition is preferably about 0.5 to 10 mass %, more preferably about 1 to 3 mass %.

### Water

Examples of water include distilled water and tap water.

The content of water in the exothermic composition is preferably about 1 to 40 mass %, more preferably about 20 to 30 mass %.

### Other Additives

In addition to iron powder, activated carbon, metal salt and water, the exothermic composition may contain other additives as required.

Examples of known additives include water-absorbing resins and water retention agents. By incorporating the water-absorbing resin, the exothermic composition can be more easily kept in a dry and smooth state. Examples of water-absorbing resins include isobutylene/maleic anhydride copolymer, polyvinyl alcohol/acrylic acid copolymer, starch/acrylate graft copolymer, polyacrylate crosslinked product, acrylate/acrylic ester copolymer, acrylate/acrylamide copolymer, and polyacrylicnitrile crosslinked product. These water-absorbing resins may be used solely, or in combination.

The particle diameter of the water-absorbing resin is set within a range that does not impair the effect of the present invention. The particle diameter is generally about 100 to 500 μm, preferably about 250 to 400 μm.

The content of the water-absorbing resin in the exothermic composition is preferably about 1 to 10 mass %, more preferably about 1 to 3 mass %.

Examples of water retention agents include wood flour, perlite, vermiculite etc.

The particle diameter of the water retention agent is set within a range that does not impair the effect of the present invention. The particle diameter is generally not more than about 300 μm, preferably not more than about 250 μm.

The content of the water retention agent in the exothermic composition is preferably about 1 to 10 mass %, more preferably about 2 to 5 mass %.

### Mixture of Components

By mixing the above components, the exothermic composition can be prepared. The mixing may be performed either in a vacuum or in the presence of inactive gas, for example, according to the method disclosed in U.S. Pat. No. 4,649,895.

### Heating Element

By sealing the exothermic composition in the bag, the heating element of the present invention is completed. The sealing is carried out, for example, by placing the exothermic composition in the bag, and closing the bag to prevent the composition from flowing out of the bag. For example, a drawstring bag-type heating element is formed by wrapping the exothermic composition with the aforementioned laminate having a circular shape, and tying the opening with a string.

The filing rate of the exothermic composition in the heating element is about 4 to 95%, preferably about 4 to 40%. A filing rate ranging from about 4 to 95% ensures desirable fitting of the heating element into the hand.

The filing rate can be found by the following equation.

A=B×100

wherein A expresses the weight (g) of the exothermic composition to be actually sealed in the bag, and B expresses the weight (g) of the maximum amount of the exothermic composition containable in the bag. The heating element is formed into an appropriate size that allows the user to easily hold it in the hand. For example, the diameter of a drawstring-type heating element is preferably about 7 to 50 cm, more preferably about 10 to 45 cm.

The weight of the exothermic composition is preferably not less than about 5 g, more preferably not less than about 15 g, further preferably in a range of about 15 to 200 g. The weight of not less than about 5 g allows the heating element to more easily fit into the hand, thereby allowing the heating element to appropriately exhibit the thermal effect. The weight of not more than about 200 g does not easily cause fatigue in the hand, thereby allowing the user to more easily perform the exercise of grasping and releasing the heating element for a long period of time.

Since the iron powder in the heating element easily reacts with oxygen in the air, the heating element is generally contained in an airtight package before distribution so as to prevent the heating element from contacting the air.

### Usage of Heating Element

When the heating element of the present invention is grasped with a hand, the heating element fits into the hand. Therefore, the heating element is suitable for an exercise of grasping and releasing the heating element. With this exercise, the exothermic composition inside the heating element generates heat, thereby keeping the temperature of the heating element generally at about 40° C. Therefore, the heating element of the present invention exhibits a thermal effect while allowing the user to perform the above exercise.

For example, the heating element of the present invention provides an effect of alleviating diseases and symptoms in hand/arm areas, including hands, fingers, and arms (preferably fingers). Further, the heating element of the present invention can be used as an instrument for rehabilitation after treatment.

The heating element of the present invention is useful for treatment of various diseases. Particularly, the heating element of the present invention is useful in treating arthralgia, including tenovaginitis and articular rheumatism.

Further, the heating element of the present invention easily fits into the target body part; therefore, the heating element is also useful for treatment of hemorrhoids, lower back pain, etc.

Further, the heating element of the present invention volatilizes moisture when it generates heat, thereby moistening the target body part. Thus, the heating element also has a moisturizing effect.

With its excellent thermal effect, the heating element of the present invention is not only useful as an instrument for treatment or rehabilitation, but also for pocket warmers for warming the body. Further, since the heating element of the present invention can more easily fit into the target body part, it is capable of warming body parts not easily accessible by existing instruments, such as the lateral surfaces of fingers.

### EFFECT OF INVENTION

The heating element of the present invention sufficiently fits into the hand when the user grasps the heating element with a hand. Therefore, the user can perform the exercise of grasping and releasing the heating element of the present invention with a hand for a long period of time.

Moreover, when the user repeats the exercise, the temperature of the heating element of the present invention is increased to about 40° C., and that same temperature is maintained. Therefore, the heating element of the present invention appropriately exhibits a thermal effect during the exercise.

Furthermore, the heating element of the present invention volatilizes moisture when it generates heat, thereby moistening the target body part. Thus, the heating element also has a moisturizing effect.

### BEST MODE FOR CARRYING OUT THE INVENTION

The following more specifically explains the present invention in reference to Examples and Comparative Examples. However, the present invention is not limited to those Examples.

In the following, the water vapor transmission rate was measured according to “Testing methods for determination of the water vapor transmission rate of moisture-proof packaging materials (dish method)” defined by JIS 20208. More specifically, the water vapor transmission rate was measured at 25±0.5° C. under a relative humidity of 90±2% using a water vapor permeability cup.

The adhesion strength was measured by a 180° peeling resistance test according to JIS 20237. More specifically, each one of the laminates obtained in the Examples and Comparative Examples was adhered to a test plate, and placed still under a relative humidity of 50±5% for 5 minutes or more. After that, the laminate was continuously peeled off at a peeling speed of 300±30 mm/min.

### Examples 1 to 12 and Comparative Examples 1 to 9

### Production of Laminate of Fibrous Sheet and Resin Film

An adhesive layer was discontinuously formed by applying an ethylene vinyl acetate resin (EVA) on a nylon woven cloth (19 cm in height×100 cm in width) having a thickness of 250 μm using a spray gun. The thickness of the adhesive layer was 5 μm.

A polyethylene film was laminated on the surface of the fibrous sheet where the adhesive layer was applied. The thickness of the film was 70 μm. The water vapor transmission rate of the film was 380 g/m²·day.

The adhesion strength of the fibrous sheet and the resin film in the laminate was 0.7 N.

In this way, a laminate having a thickness of 350 μm was produced.

Preparation of Exothermic Composition

An exothermic composition was prepared by mixing iron powder having a particle diameter of 50 μm, activated carbon having a particle diameter of 200 μm, sodium chloride having a particle diameter of 380 μm, water, vermiculite having a diameter of 100 and polysodium acrylate having a diameter of 380 μm. In the exothermic composition, the contents of iron powder, activated carbon, sodium chloride, water, vermiculite and polysodium acrylate were 55 mass %, 13 mass %, 1 mass %, 26 mass %, 3 mass % and 2 mass %, respectively.

According to the aforementioned method, the exothermic composition was prepared.

### Production of Heating Element

The aforementioned laminate was cut into multiple circles so that the circles had diameters 2 cm greater than the diameters specified in Tables 1 to 4. The exothermic composition was placed on the polyethylene film side of each circle. Thereafter, in the same atmosphere, each exothermic composition was wrapped with the corresponding circular laminate, and the wrapped laminate was tied with a string at a portion 2 cm from the end.

As such, drawstring bag-shaped heating elements were produced.

Tables 1 to 4 show weights (g) and filing rates (%) of the exothermic compositions contained in the heating elements.

Each of the obtained heating elements was contained in a bag made of a polyvinylidene chloride-coated polypropylene film (KOP), and the bag was sealed to prevent the heating element from contacting air.

The following Test Examples 1 to 2 were performed immediately after each heating element was taken out from the bag made of a polyvinylidene chloride-coated polypropylene film.

### Test Example 1

A test was conducted with 10 healthy people in order to check sufficient fitting of a heating element during a five-minute exercise in which the heating element was repeatedly grasped and released. The test was performed on each of the heating elements obtained in Examples 1 to 12 and Comparative Examples 1 to 9.

A: 7 or more people thought that the heating element sufficiently fit into the hand.

B: 5 to 6 people thought that the heating element sufficiently fit into the hand.

C: 4 or less people thought that the heating element sufficiently fit into the hand.

Tables 1 to 4 show the results.

### Test Example 2

A test was conducted with 10 healthy people in order to check the thermal effect of a heating element during a five-minute exercise in which the heating element was repeatedly grasped and released. The test was performed on each of the heating elements obtained in Examples 1 to 12 and Comparative Examples 1 to 9.

A: 7 or more people sensed moderate warmth at around 40° C.

B: 5 to 6 people sensed moderate warmth at around 40° C.

C: 4 or less people sensed moderate warmth at around 40° C.

Tables 1 to 4 show the results.

TABLE 1

| Examples | 1 | 2 | 3 | 4 | 5 | 6 |
|---|---|---|---|---|---|---|
| Diameter (cm) | 10 | 10 | 10 | 20 | 20 | 20 |
| Weight (g) | 5 | 15 | 35 | 10 | 65 | 190 |
| Filing rate | 11.1 | 33.3 | 77.7 | 4.3 | 28.2 | 82.6 |
| Results of Test | A | A | B | A | A | B |
| Example 1 | | | | | | |
| Results of Test | B | A | A | B | A | A |
| Example 2 | | | | | | |

TABLE 2

| Examples | 7 | 8 | 9 | 10 | 11 | 12 |
|---|---|---|---|---|---|---|
| Diameter (cm) | 30 | 30 | 30 | 40 | 40 | 40 |
| Weight (g) | 20 | 115 | 345 | 30 | 165 | 500 |
| Filing rate | 5.2 | 30.3 | 90.7 | 5.4 | 29.7 | 90 |
| Results of Test | A | A | B | A | A | B |
| Example 1 | | | | | | |
| Results of Test | A | A | A | A | A | A |
| Example 2 | | | | | | |

TABLE 3

Comparative Examples

| | 1 | 2 | 3 | 4 |
|---|---|---|---|---|
| Diameter (cm) | 10 | 20 | 20 | 30 |
| Weight (g) | 45 | 5 | 230 | 10 |
| Filing rate | 100 | 2.17 | 100 | 2.6 |
| Results of Test | C | C | C | C |
| Example 1 | | | | |
| Results of Test | A | B | A | B |
| Example 2 | | | | |

TABLE 4

| Comparative Examples | 5 | 6 | 7 | 8 | 9 |
|---|---|---|---|---|---|
| Diameter (cm) | 30 | 40 | 40 | 45 | 45 |
| Weight (g) | 380 | 15 | 555 | 20 | 650 |
| Filing rate | 100 | 2.7 | 100 | 3 | 100 |
| Results of Test | C | C | C | C | C |
| Example 1 | | | | | |
| Results of Test | A | A | A | A | A |
| Example 2 | | | | | |

### Examples 13 to 21

Each drawstring bag-shaped heating element was produced using the same method as in Example 5, except that the adhesion strength of the fibrous sheet and the resin film in the laminate was set to the values specified in Tables 5 to 6.

### Comparative Example 10

A drawstring bag-shaped heating element was produced using the same method as in Example 5, except that the polyethylene film was not laminated on the nylon woven cloth. More specifically, instead of the aforementioned laminate, the nylon woven cloth was used as the bag for wrapping the exothermic composition.

### Comparative Example 11

A drawstring bag-shaped heating element was produced using the same method as in Example 5, except that the polyethylene film was used instead of the aforementioned laminate as the bag for wrapping the exothermic composition.

### Comparative Example 12

A drawstring bag-shaped heating element was produced using the same method as in Example 5, except that the adhesive layer was not formed on the nylon woven cloth. More specifically, instead of the aforementioned laminate, the bag for wrapping the exothermic composition was made by simply overlaying the polyethylene film on the nylon woven cloth.

### Comparative Examples 13 to 14

Each drawstring bag-shaped heating element was produced using the same method as in Example 5, except that the adhesion strength of the fibrous sheet and the resin film in the laminate was set to the values specified in Table 7.

Each of the heating elements obtained in Examples 13 to 21 and Comparative Examples 10 to 14 was contained in a bag made of a polyvinylidene chloride-coated polypropylene film (KOP), and the bag was sealed to prevent the heating element from contacting air.

The following Test Examples 3 to 5 were performed immediately after each heating element was taken out from the bag made of a polyvinylidene chloride-coated polypropylene film.

### Test Example 3

A test was conducted with 10 healthy people in order to check sufficient fitting of a heating element during a five-minute exercise in which the heating element was repeatedly grasped and released. The test was performed on each of the heating elements obtained in Examples 13 to 21 and Comparative

### Examples 10 to 14

A: 7 or more people thought that the heating element sufficiently fit into the hand.

B: 5 to 6 people thought that the heating element sufficiently fit into the hand.

C: 4 or less people thought that the heating element sufficiently fit into the hand.

Tables 5 to 7 show the results.

### Test Example 4

A test was conducted with 10 patients suffering from arthralgia caused by tenovaginitis in order to check sufficient fitting of each heating element during a five-minute exercise in which the heating element was repeatedly grasped and released. The test was performed on each of the heating elements obtained in Examples 15 to 18 and Comparative Examples 13 and 14.

A: 7 or more people thought that the heating element sufficiently fit into the hand.

B: 5 to 6 people thought that the heating element sufficiently fit into the hand.

C: 4 or less people thought that the heating element sufficiently fit into the hand.

Table 8 shows the results.

### Test Example 5

A test was conducted with 10 patients suffering from arthralgia caused by tenovaginitis in order to check the pain alleviation effect of a heating element after a five-minute exercise in which the heating element was repeatedly grasped and released. The test was performed on each of the heating elements obtained in Examples 15 to 18 and Comparative Examples 13 and 14.

A: 7 or more people thought that the pain was alleviated.

B: 5 to 6 people thought that the pain was alleviated.

C: 4 or less people thought that the pain was alleviated.

Table 8 shows the results.

TABLE 5

| Examples | 13 | 14 | 15 | 16 | 17 |
|---|---|---|---|---|---|
| Adhesion | 0.3 | 0.5 | 1 | 1.5 | 2 |
| strength | | | | | |
| (N) | | | | | |
| Results of | A | A | A | A | A |
| Test | | | | | |
| Example 3 | | | | | |

TABLE 6

Examples

| | 18 | 19 | 20 | 21 |
|---|---|---|---|---|
| Adhesion | 2.5 | 3 | 3.5 | 5 |
| strength | | | | |
| (N) | | | | |
| Results of | A | A | B | B |
| Test | | | | |
| Example 3 | | | | |

TABLE 7

| Examples | 10 | 11 | 12 | 13 | 14 |
|---|---|---|---|---|---|
| Adhesion | — | — | 0 | 7 | 10 |
| strength | | | | | |
| (N) | | | | | |
| Results of | C | C | C | C | C |
| Test | | | | | |
| Example 3 | | | | | |

TABLE 8

| | |
|---|---|
| | Comparative |
| Examples | Examples |

| | 15 | 18 | 13 | 14 |
|---|---|---|---|---|
| Adhesion | 1 | 2.5 | 7 | 10 |
| strength (N) | | | | |
| Results of | A | A | C | C |
| Test Example 4 | | | | |
| Results of | A | A | C | C |
| Test Example 5 | | | | |

### Test Example 6

A test was conducted with 10 people who have dry hands in order to check the moistening effect of a heating element after a five-minute exercise in which the heating element was repeatedly grasped and released. The test was performed on each of the heating elements obtained in Examples 1 to 21.

All of the 10 test subjects evaluated the heating elements obtained in Examples 1 to 21 as having moistening effects.

### BRIEF DESCRIPTION OF DRAWINGS

FIG. 1 illustrates a drawstring bag-shaped heating element as an example of the heating element of the present invention.

FIG. 2 is an upper view of a laminate for fabricating the heating element of the present invention.

FIG. 3 is a cross-sectional view of a laminate for fabricating the heating element of the present invention.

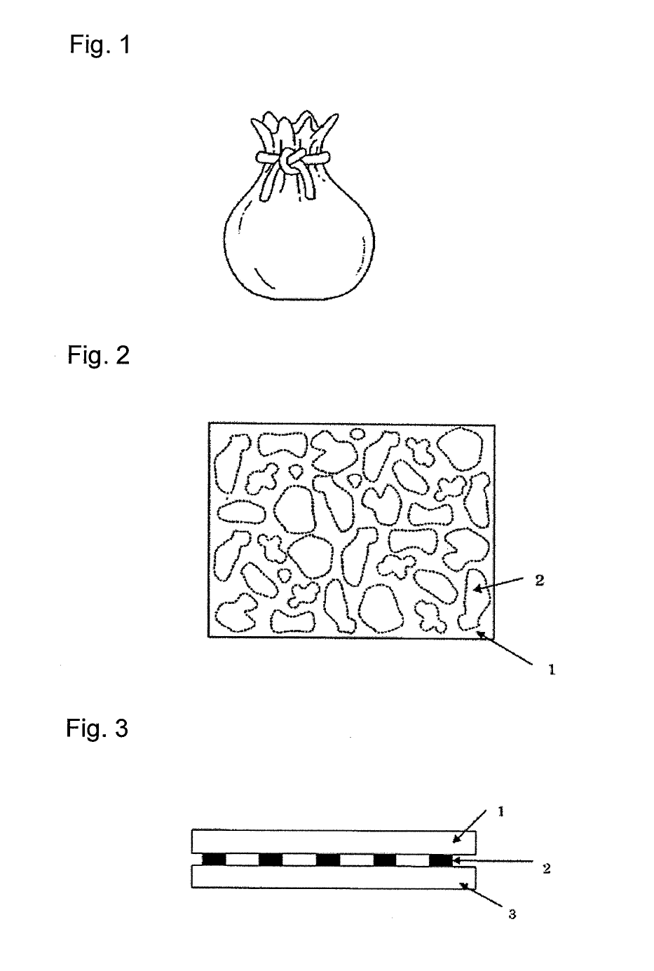

### REFERENCE NUMERALS

1. Resin film
2. Adhesive layer
3. Fibrous sheet

1. A heating element comprising an exothermic composition that generates heat by contact with air, the exothermic composition being sealed in an air-permeable bag that has an outer face made of a fibrous sheet,
wherein:
(1) the air-permeable bag is formed of a laminate obtained by layering a resin film on the fibrous sheet,
(2) the laminate is formed by discontinuously bonding the fibrous sheet and the resin film, and
(3) the filing rate of the exothermic composition ranges from 4 to 95%.

2. The heating element according to claim 1, wherein the fibrous sheet and the resin film in the laminate are bonded at an adhesion strength of from 0.2 to 6 N.

3. The heating element according to claim 1, wherein an adhesive layer is discontinuously formed between the fibrous sheet and the resin film.

4. The heating element according to claim 1, wherein the air-permeable bag has a spherical or cylindrical shape.

5. The heating element according to claim 1, wherein the fibrous sheet is a nonwoven or woven cloth of natural or artificial fiber.

6. The heating element according to claim 1, wherein the exothermic composition is a composition containing iron powder, activated carbon, metal salt and water, and wherein the contents of the iron powder, the activated carbon, the metal salt and the water are 30 to 80 mass %, 3 to 25 mass %, 0.5 to 10 mass % and 1 to 40 mass %, respectively.

7. The heating element according to claim 1, wherein the resin film contains, as a resin component, at least one synthetic resin selected from the group consisting of polyethylene, polypropylene, nylon polyester and polyvinyl chloride.

8. The heating element according to claim 1, wherein the heating element is an instrument for treatment or rehabilitation.
